(19) United States
(12) Patent Application Publication
(10) Pub. No.: US 2022/0151359 A1
(43) Pub. Date: May 19, 2022

(54) SANITIZING LUGGAGE

(71) Applicant: simplehuman, LLC, Torrance, CA (US)

(72) Inventor: Frank Yang, Rancho Palos Verdes, CA (US)

(21) Appl. No.: 17/524,376

(22) Filed: Nov. 11, 2021

Related U.S. Application Data
(60) Provisional application No. 63/113,743, filed on Nov. 13, 2020.

(51) Int. Cl. A45C 13/02; A45C 5/14; A61L 2/18

(52) U.S. Cl. CPC A45C 13/02; A45C 5/14; A61L 2202/182; A61L 2202/15; A61L 2202/16; A61L 2/18

(57) ABSTRACT

This disclosure includes a description of luggage devices configured to provide convenient access to sanitizers. The luggage device can include a receptacle having an opening to receive a sanitizer container therein. The luggage device can include a support that is translated or moved within the receptacle by way of a push-to-release mechanism. The push-to-release mechanism can translate or move the support between a deployed position in which the sanitizer container is at least partially exposed outside the receptacle and a stowed position in which the sanitizer container is disposed inside the receptacle. The luggage device can include a dispenser that enables the user to dispense sanitizer from a sanitization container in the receptacle.

### RELATED APPLICATIONS

This application claims the benefit under 35 U.S.C. § 119(e) of U.S. Provisional Patent Application No. 63/113,743, filed on Nov. 13, 2020, which is hereby incorporated by reference in its entirety herein and made a part of this specification. Any and all applications for which a foreign or domestic priority claim is identified in the Application Data Sheet as filed with the present application are hereby incorporated by reference under 37 CFR 1.57.

### BACKGROUND

### Field

This disclosure relates generally to sanitization devices and methods, and specifically to sanitization containers.

### Description of the Related Art

People spread microbes and contract diseases, viruses, and the like therefrom by way of their hands. For example, a person's hand may touch a public surface, transferring microbes from the public surface to the person's hand. However, a person will often not contract a disease, virus, or the like from merely touching the public surface having microbes. Instead, the person may contract a disease, virus, or the like by way of touching the person's face after touching the microbe infested public surface, introducing microbes into the person's body via the mouth, nose, or eyes. The spread of microbes and contraction of diseases, viruses, and the like therefrom can be diminished by cleansing a person's hands.

### SUMMARY

This disclosure includes a description of a luggage device. The luggage device can include a receptacle having an opening. The receptacle can receive a sanitizer container through the opening. The luggage device can include a support that can support the sanitizer container within the receptacle. The luggage device can include a releasable holder, such as a push-to-release mechanism, that can translate or move the support between a deployed position in which the sanitizer container is at least partially exposed outside the receptacle and a stowed position in which the sanitizer container is disposed inside the receptacle.

In some embodiments, a luggage device can dispense a sanitizer from a sanitization container disposed within a receptacle. The luggage device can include a receptacle with an opening. The receptacle can receive a sanitizer container through the opening. The luggage device can include a dispenser that can be fluidically coupled to the sanitizer container in the receptacle and extend outside the receptacle. The luggage device can include a pumping assembly. The pumping assembly can be operatively coupled to the dispenser. The pumping assembly can pump a sanitizer in the sanitizer container out of the dispenser upon an application of a pushing force on the dispenser. The dispenser can be configured to be manipulated between a deployed configuration and a stowed configuration.

### BRIEF DESCRIPTION OF THE DRAWINGS

These drawings are schematic, showing some examples of basic parts and concepts. Many different or additional structures, implementations, components, mechanisms, steps, and processes can be used. The claimed inventions should not be limited in any way to anything illustrated in the drawings.

FIG. 1 is an illustration of a luggage device having a receptacle with a sanitizer container therein.

FIG. 2 is an illustration of an example of FIG. 1 wherein the receptacle is disposed between arms of a handle.

FIGS. 3A and 3B are illustrations of another example of FIG. 1 wherein a cover can rotate between open and closed configurations, covering and uncovering an opening of the receptacle.

FIGS. 4A and 4B are illustrations of another example of FIG. 1 wherein a cover can slide between open and closed configurations, covering and uncovering an opening of the receptacle.

FIGS. 5A-5C are illustrations of another example of FIG. 1 wherein a push-to-release mechanism can move the sanitizer container within the receptacle between stowage and access configurations.

FIG. 5D is an illustration of an example of FIGS. 5A-5C wherein the push-to-release mechanism can be deployed via manipulation of a button.

FIGS. 6A and 6B are illustrations of another example of FIG. 1 wherein a compartment that can receive the sanitizer container therein can move within the receptacle between stowed and deployed configurations.

FIG. 6C is an illustration of an example of FIGS. 6A and 6B wherein the push-to-release mechanism can be deployed via manipulation of a button.

FIG. 7 is an illustration of another example of FIG. 1 wherein a dispenser facilitates dispensing sanitizer from the sanitization container while positioned in the receptacle.

FIG. 8 is an example of FIG. 7 wherein the receptacle is positioned between arms of a handle.

FIGS. 9A-9C are illustrations of another example of FIG. 7 wherein the dispenser can be rotated between deployed and stowed configurations.

FIGS. 10A-10C are illustrations of another example of FIG. 7 wherein the dispenser can be twisted between deployed and stowed configurations.

FIGS. 11A-11C are illustrations of another example of FIG. 7 wherein the dispense can be automatically moved from a stowed configuration to a deployed configuration by way of manipulating a button.

### DETAILED DESCRIPTION

This specification provides textual descriptions and illustrations of many devices. Any structure, material, function, method, or step that is described and/or illustrated in one example can be used by itself or with or instead of any structure, material, function, method or step that is described and/or illustrated in another example or used in this field. The text and drawings merely provide examples and should not be interpreted as limiting or exclusive. No feature disclosed in this application is considered critical or indispensable. The relative sizes and proportions of the components illustrated in the drawings form part of the supporting disclosure of this specification, but should not be considered to limit any claim unless recited in such claim.

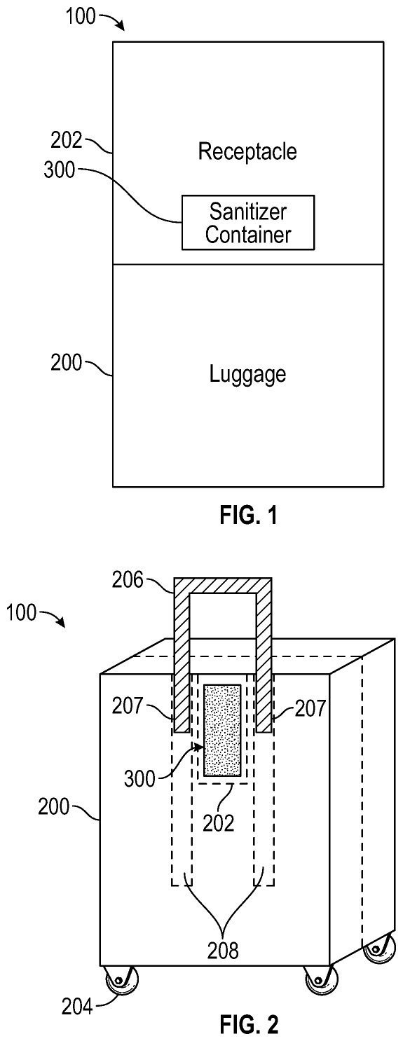

As illustrated in FIG. 1, in some embodiments a sanitizing luggage 100, also referred to as a luggage device or system, can comprise a luggage 200 having a receptacle 202 configured to receive a sanitizer container 300 therein. In general, as used herein, luggage 200 is a person-carried receptacle for transporting one or more items. Some examples of luggage 200 can at least include a suitcase, baggage, container, tote, trunk, bag, carry-on, box, computer case, backpack, purse, satchel, messenger bag, briefcase, fanny pack, etc. The receptacle 202 can be disposed within the luggage 200 or coupled thereto. Some examples of the receptacle 202 can at least include a cavity, compartment, holder, hollow, hole, chamber, etc. An opening of the receptacle 202 can be closed and opened via a cover. In some variants, the receptacle 202 can include a rigid periphery, boundary, frame, structure, or features to protect the content(s) therein. For example, in some variants, the receptacle 202 and/or features thereof can be made of a polymer (such as plastic), metal (such as aluminum), metal alloy, ceramic, and/or other material. In some variants, the receptacle 202 and/or features thereof can be flexible or pliable.

The sanitizer container 300 can be removably stowed within the receptacle 202 for convenient transportation and access. For example, the sanitizer container 300 can be placed in the receptacle 202 when not in use and removed therefrom when desired. Some examples of the sanitizer container 300 can at least include a pod, bottle, capsule, can, carton, pouch, vessel, vial, package, bag, tank, jar, tube, etc. In some variants, the receptacle 202 can include a cover that can be opened, providing access to the sanitizer container 300, and closed, securing, covering, and/or protecting the sanitizer container 300 within the receptacle 202. In some variants, the cover of the receptacle 202 can be locked in place, preventing access therein without a key, combination, biometric key, etc.

In some situations, the complete removability and/or separation of the sanitizer container 300 can be helpful, such as with air travel. For example, some agencies require the removal and inspection of containers for liquids, gels, etc. when traveling by air. Accordingly, the sanitizer container 300 can be removed for such inspections. In some embodiments, the receptacle 202 can be configured to receive sanitizer containers 300 of various sizes. In some embodiments, the receptacle 202 can be configured to receive a specific size and/or shape of sanitizer container 300, which may comply with an agency regulation for air travel. For example, the United States Transportation Security Administration (USTSA) currently limits the volume of carry-on liquids for air travel to be less than or equal to about 100 mL (about 3.4 fluid ounces). In some embodiments, the volume of the receptacle 202 can be selected to comply with this or any other regulation of the USTSA or any other government entity or agency, security company, airline, and/or other travel company, etc. that is currently in force or implemented in the future. The receptacle 202 can be configured to have a variable volume and/or one or a series of measuring lines to demonstrate compliance with an applicable regulation.

In some variants, the receptacle 202 can be quickly and easily removable from the luggage 200 by a consumer without requiring the use of tools. In some variants, the receptacle 202 is fixedly attached or integrated with the luggage 200.

The sanitizer container 300 can hold a sanitizer therein. Some examples of the sanitizer include at least any biocompatible antimicrobial fluids (e.g., liquid, gel, or gas) or other substances, such as alcohol, chlorhexidine, iodine, bleach, chlorine, nitric oxide, and/or metal ions (e.g., silver or copper ions), etc. In some embodiments, an antimicrobial-emitting substance can be used. In some embodiments, the sanitizer container 300 can be a dispenser of antimicrobial wipes, such as wipes that include one or more antimicrobial fluids or other substances, including any of those described in this specification or otherwise. Any other antimicrobial fluid or substance can be used. The sanitizer container 300 can, in some embodiments, be disposable. The sanitizer container 300 can, in some embodiments, be refillable with sanitizer and reusable.

In any embodiment in this specification, the sanitizing luggage 100, luggage 200, receptacle 202, and/or sanitizer container 300 can comprise one or more antimicrobial features. For example, in some embodiments, any or all of the sanitizing luggage 100, luggage 200, receptacle 202, and/or sanitizer container 300 can comprise an antimicrobial texture and/or an antimicrobial coating or covering.

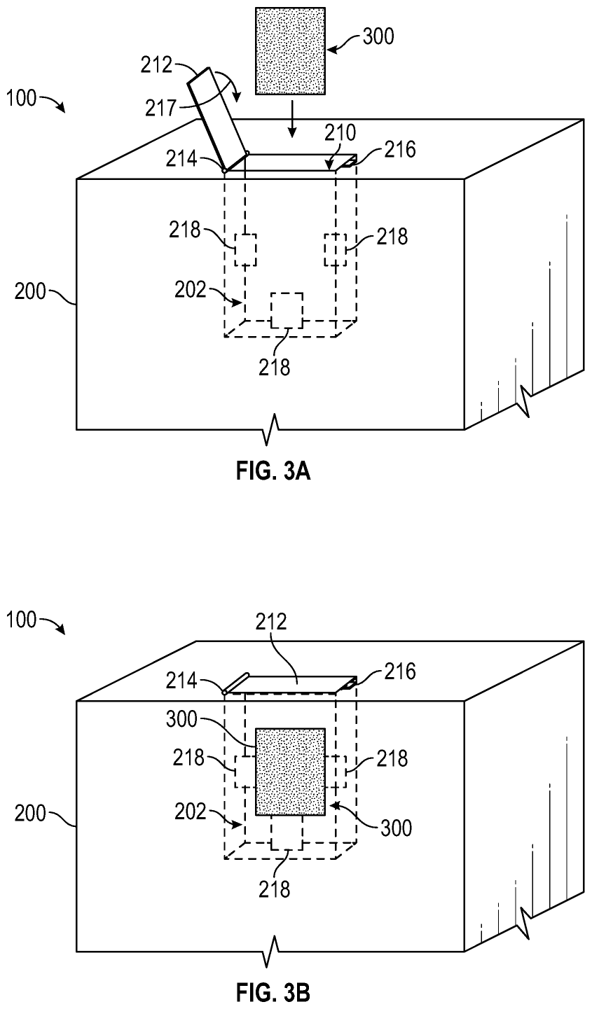

FIG. 2 shows an example of the sanitizing luggage 100 with the receptacle 202 conveniently disposed between arms 207 of a handle 206 of the luggage 200. In some embodiments, the arms 207 of the handle 206 can be telescoping (e.g., between an extended or retracted position) such that the arms 207 can collapse and expand in and out of the cavities 208, e.g., voids, chambers, slots, etc., of the luggage 200 to move the handle 206. The region between the arms 207 can be rigid, providing structural support and/or protection for the receptacle 202. For example, the cavities 208, arms 207, and/or surrounding structure of the luggage 200 can protect the receptacle 202 from collapsing under a load. The region between the arms 207 can be in close proximity to a user's hand when moving the sanitizing luggage 100 via the handle 206 (e.g., rolling via the wheels 204 by pulling on the handle 206) to facilitate quick access. In some embodiments, the receptacle 202 can be positioned at any other positions on the luggage 200.

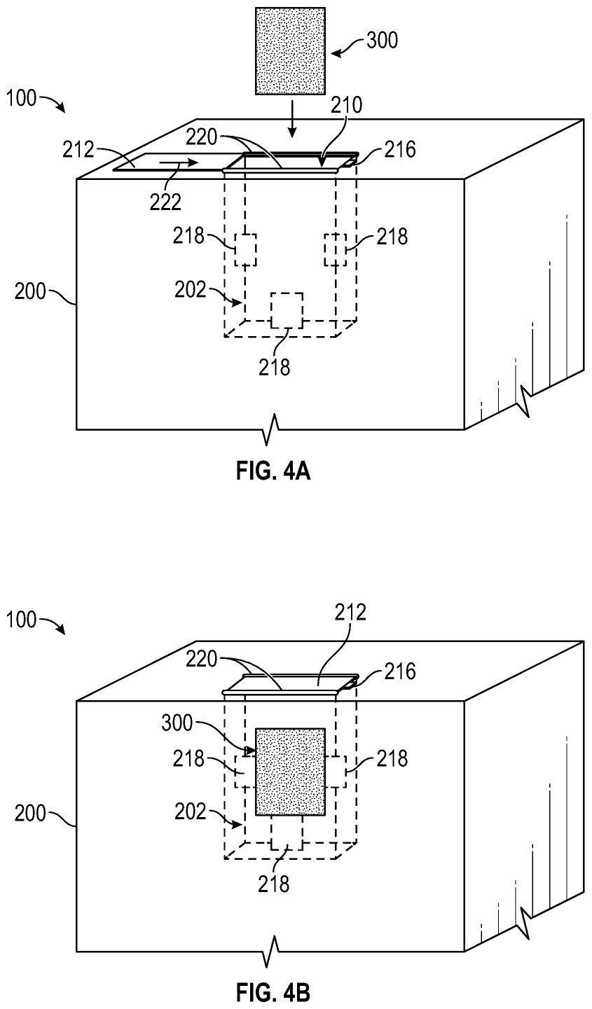

FIGS. 3A and 3B show an example of the sanitizing luggage 100 with a cover 212 that can rotate between open and closed configurations, covering and uncovering an opening 210 providing access into the receptacle 202. FIG. 3A illustrate the cover 212 rotated to an open position such that the opening 210 is uncovered and accessible by a user. With the opening 210 uncovered and accessible, the sanitizer container 300 can be placed into the receptacle 202. The cover 212 can be rotated about a hinge 214, which may also be referred to as a pivot or joint, in the direction of arrow 217 to cover the opening 210, as shown in FIG. 3B. The cover 212 can be secured via a lock 216. The lock 216 can be a latch, clip, fastener, hook, clamp, clasp, catch, bracket, etc. that engages with the cover 212. In some variants, the cover 212 can be zipped to cover the opening 210, which can include joining the periphery of the cover 212 to the periphery of the opening 210 by way of a zipper. The cover 212 and opening 210 can be disposed on any side of the luggage 200, which can at least include a top and one or more lateral sides of the luggage 200. The cover 212 can be rigid or flexible. The cover 212 can be rotated open by way of unlocking the lock 216 and rotating the cover 212 in a direction that is opposite the arrow 217.

The receptacle 202 can include one or more retainers 218 that are configured to secure the sanitizer container 300 within the receptacle 202, which can include reducing movement of the sanitizer container 300 within the receptacle 202 during travel. The one or more retainers 218 can engage the periphery of the sanitizer container 300 to substantially hold or help secure the sanitizer container 300 in place during travel. In some variants, the one or more retainers 218 are made of a compressible or elastomeric material, such as foam or rubber, such that sanitizer container 300 of varying sizes and shapes can be secured in position by the one or more retainers 218 and/or to securely receive the sanitizer container 300. Some examples of the retainers 218 include at least tabs, blocks, flanges, hook-and-loop fastener (e.g., Velcro fastener), etc. In some embodiments, the one or more retainers 218 can include one or more springs that bias a surface of the retainers 218 against the sanitizer container 300 to increase friction against the sanitizer container 300. In some variants, one or more retainers 218 can be disposed on an inner surface of the cover 212.

In some variants, the receptacle 202 does not include one or more retainers 218. In some variants, the receptacle 202 is configured to receive a sanitizer container 300 of a particular size and/or shape. For example, the periphery of the sanitizer container 300 can be proximate the periphery of the receptacle 202 such that the sanitizer container 300 is not free to substantially move when positioned within the receptacle 202. The receptacle 202 can be various sizes and shapes. For example, the receptacle 202 can be in the shape of a generally rectangular prism with the opening 210 providing access therein. The receptacle 202 can be other shapes such as a cylinder. The receptacle 202 can have a cross-section that is polygonal (e.g., triangular, square, rectangular, pentagonal, hexagonal, etc.), circular, oval, irregular, and/or others.

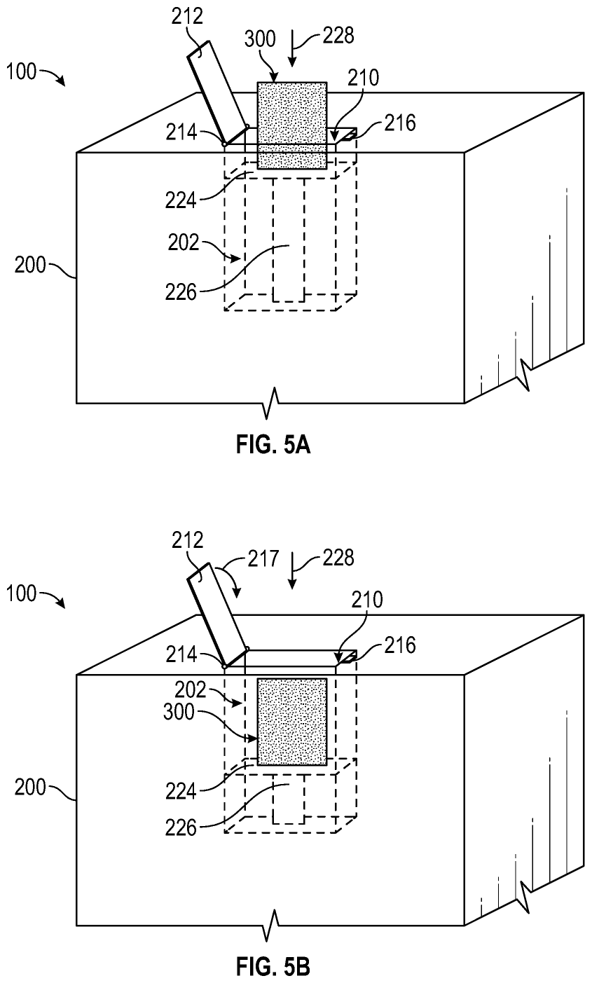

FIGS. 4A and 4B show an example of the sanitizing luggage 100 with a cover 212 that can slide between open and closed configurations, covering and uncovering the opening 210 providing access into the receptacle 202. FIG. 4A illustrates the cover 212 translated (e.g., slid or otherwise moved) open such that the opening 210 is uncovered. The cover 212 can be slid in the direction of arrow 222 to cover the opening 210, as shown in FIG. 4B. The cover 212 can be rigid to facilitate translation of the cover 212. The sanitizing luggage 100 can include rails 220 that guide the translation of the cover 212. The rails 220 can be channels, slots, guides, grooves, etc. The cover 212 can be secured by way of the lock 216, as described elsewhere herein. To uncover the opening 210, the lock 216 can be unlocked and the cover 212 may be translated or moved in a direction that is opposite the arrow 222.

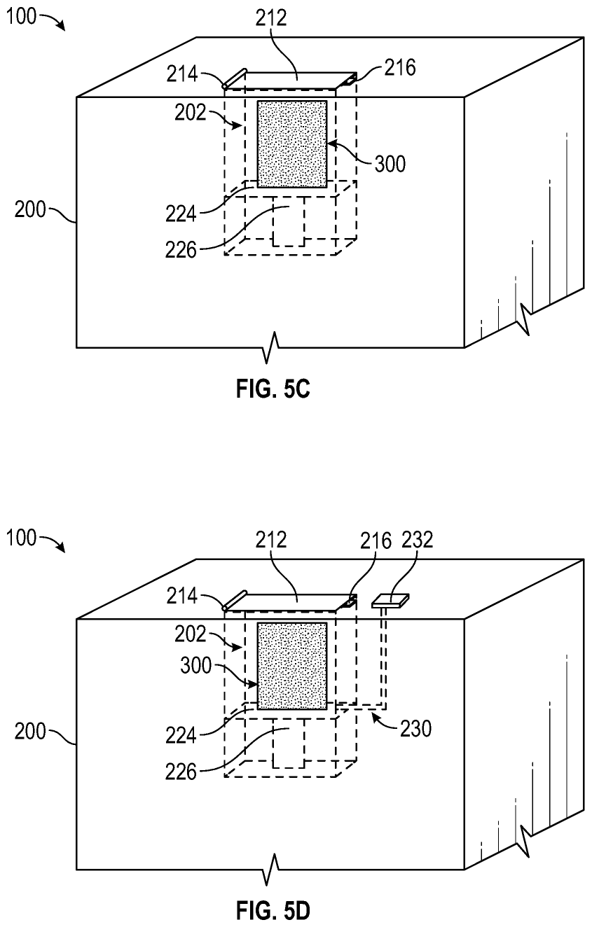

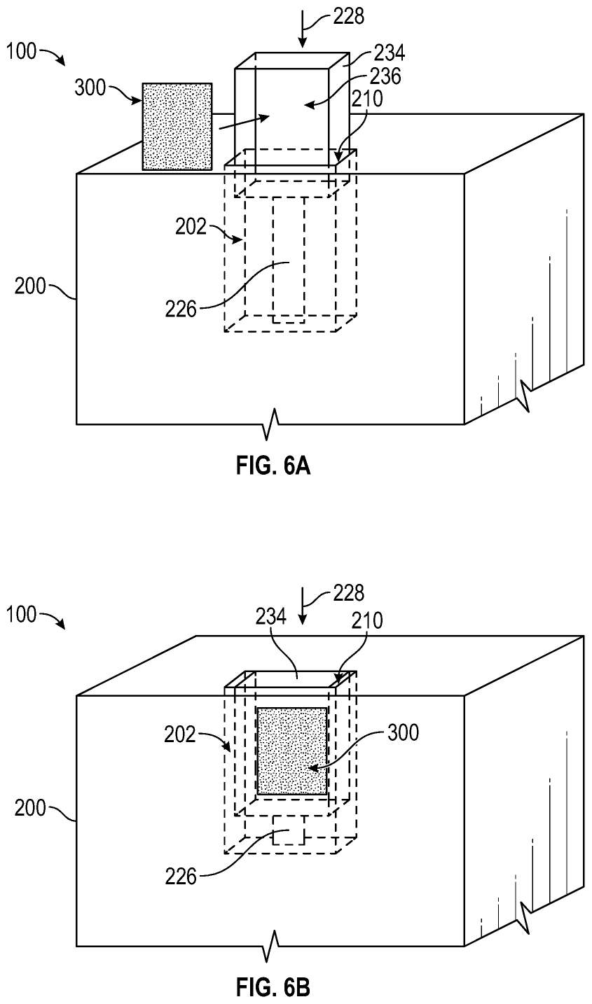

FIGS. 5A-5C show an example of the sanitizing luggage 100 with a releasable holder, such as a push-to-release mechanism 226, that can translate or move a support 224 to move the sanitizer container 300 between stowed and deployed configurations. The push-to-release mechanism 226 may be a push-open mechanism, touch-release lock spring, push-push mechanism, etc. The push-to-release mechanism 226 may be pushed into a stowed position, as illustrated in FIG. 5B, and retained therein until pushed again, releasing the push-to-release mechanism 226 to extend to a deployed position such that the sanitizer container 300 is readily accessible.

FIG. 5A illustrates the cover 212 rotated open and the sanitizer container 300 placed on the support 224. The support 224 may also be referred to as a platform, floor, panel, etc. The sanitizer container 300 may be pushed in the direction of arrow 228, moving the sanitizer container 300 and support 224 in the direction of arrow 228 as the push-to-release mechanism 226 collapses, translates, telescopes, and/or otherwise moves until arriving at a stowed configuration—an example of which is illustrated in FIG. 5B. In the stowed configuration, the push-to-release mechanism 226 can retain support 224 at a stowed position such that the sanitizer container 300 is positioned inside the receptacle 202. The push-to-release mechanism 226 can include one or more clips, springs, clamps, latches, hooks, etc. that can retain the push-to-release mechanism 226 and support 224 in the stowed position, as illustrated in FIG. 5B. With the push-to-release mechanism 226, support 224, and sanitizer container 300 in the stowed configuration, the cover 212 can be rotated in the direction of arrow 217 about hinge 214 to cover the opening 210, as illustrated in FIG. 5C. As described herein, a lock 216 can secure the cover 212 over the opening 210 to secure the sanitizer container 300 within the receptacle 202. In some embodiments, the cover 212 can zip shut with the periphery defining the opening 210.

To access the sanitizer container 300, the lock 216 may be unlocked, allowing the cover 212 to be rotated open in a direction opposite the direction of arrow 217 about the hinge 214 to expose the sanitizer container 300. The sanitizer container 300 may be pushed again in the direction of arrow 228, releasing the push-to-release mechanism 226 to deploy to a deployed position—an example of which is illustrated in FIG. 5A. The push-to-release mechanism 226 can include one or more springs biasing the push-to-release mechanism 226 toward the deployed configuration. The one or more clips, arms, springs, clamps, latches, hooks, etc. of the push-to-release mechanism 226 can be moved to allow the one or more springs to deploy the push-to-release mechanism 226 toward the deployed configuration upon the sanitizer container 300 being pushed in the direction of arrow 228 and then released. With the push-to-release mechanism 226 and support 224 in the deployed configuration, the sanitizer container 300 can be positioned at least partially outside the receptacle 202 and/or proximate the opening 210, enabling the sanitizer container 300 to be easily grasped.

FIG. 5D illustrates an example of the sanitizing luggage 100 wherein the push-to-release mechanism 226 can be deployed via manipulation of a button 232 that maneuvers an arm 230. The arm 230 can engage with the support 224 and/or push-to-release mechanism 226 to prevent the one or more springs of the push-to-release mechanism 226 from moving the push-to-release mechanism 226 and support 224 toward the deployed position. The arm 230 can automatically engage the support 224 and/or push-to-release mechanism 226 upon the support 224 and/or push-to-release mechanism 226 being placed in the stowed configuration. For example, the arm 230 can be biased toward engagement, which can be via one or more springs. The arm 230 can be disengaged from the support 224 and/or push-to-release mechanism 226 upon the manipulation of the button 232, which can allow the one or more springs of the push-to-release mechanism 226 to extend the push-to-release mechanism 226 to translate or move the support 224 to the deployed position such that the sanitizer container 300 is at least partially outside the receptacle 202 and/or proximate the opening 210. Some examples of the arm 230 can at least include a clip, spring, clamp, latch, hook, etc. In some variants, more than one arm 230 is employed. In any embodiment, the sanitizer of the sanitizing luggage 100 can be a removable or integrated electronic sanitizing system configured to automatically dispense or emit a sanitizing substance on a user's hands, such as using an electrical pump or sprayer system. The electronic sanitizing system can include an electrically powered pump or sprayer, a battery, an electronic processor, one or more electrical switches, and/or one or more electronic proximity sensors, etc. For example, in some embodiments, the electronic sanitizing system can include or be used with or instead of any structure, material, function, method or step that is described and/or illustrated in U.S. Pat. No. 8,678,244, which is incorporated by reference herein in its entirety and made a part of this specification.

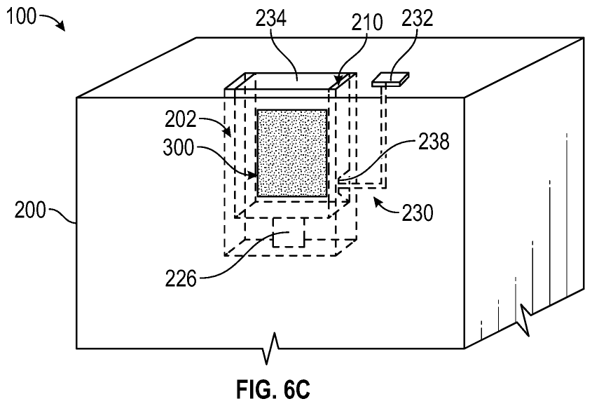

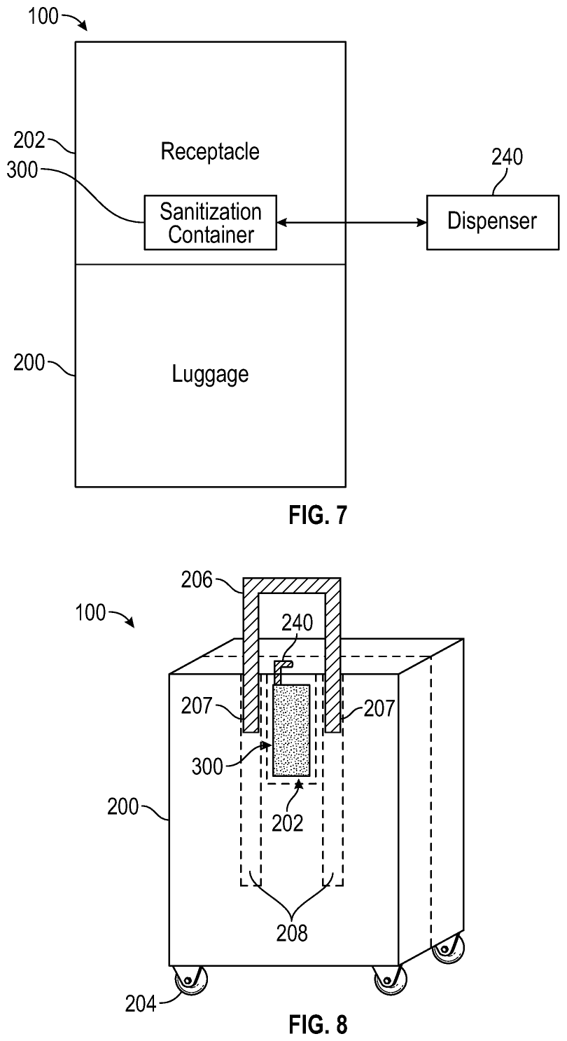

FIGS. 6A and 6B show an example of the sanitizing luggage 100 wherein a compartment 234, that can receive the sanitizer container 300, can be moved within the receptacle 202 by way of the push-to-release mechanism 226 between stowed and deployed configurations. The compartment 234 can include an opening 236 through which the sanitizer container 300 may be received. The compartment 234 may also be referred to as a holder or chamber. The compartment 234 can be configured to receive a sanitizer container 300 of a particular size and/or shape. In some embodiments, the compartment 234 can be configured to receive sanitizer containers 300 of varying sizes and/or shapes. In some embodiments, the compartment 234 can include one or more retainers 218, as described herein. The compartment 234 can be in the shape of a rectangular prism that is hollow with the opening 236 providing access therein. The compartment 234 can be other shapes such as a cylinder. The compartment 234 can have a cross-section that is polygonal (e.g., triangle, square, rectangle, pentagon, hexagon, etc.), circular, oval, irregular, and/or others.

The compartment 234 can be pushed in the direction of arrow 228 to place the compartment 234 in the stowed configuration, as illustrated in FIG. 6B. The push-to-release mechanism 226 can collapse, translate, telescope, and/or otherwise move the compartment 234 from the deployed configuration to the stowed configuration. As described herein, the push-to-release mechanism 226 can retain the compartment 234 in the stowed configuration, which can be via one or more clips, arms, springs, clamps, latches, hooks, etc. In the stowed configuration, the opening 236 can be positioned within the receptacle 202 impeding access to the sanitizer container 300 and/or unintended removal of the sanitizer container 300 from within the receptacle 202. To deploy the push-to-release mechanism 226, a force can be applied in the direction of arrow 228 to the compartment 234, allowing one or more springs of the push-to-release mechanism 226 to deploy the push-to-release mechanism 226 such that the compartment 234 is moved at least partially outside of the receptacle 202. As described herein, applying a pushing force in the direction of arrow 228 to the compartment 234 while the compartment 234 is positioned within the receptacle 202, e.g., in the stowed configuration, can maneuver one or more clips, arms, springs, clamps, latches, hooks, etc. to disengage to allow the one or more springs to deploy the push-to-release mechanism 226.

FIG. 6C illustrates an example of the sanitizing luggage 100 wherein the push-to-release mechanism 226 can be deployed by way of manipulation of the button 232 that maneuvers an arm 230. The arm 230 can engage with the compartment 234 and/or push-to-release mechanism 226 to prevent the one or more springs of the push-to-release mechanism 226 from moving the push-to-release mechanism 226 and compartment 234 toward the deployed position, similar to the description in reference to FIG. 5D. The arm 230 can automatically engage the compartment 234 and/or push-to-release mechanism 226 upon the compartment 234 and/or push-to-release mechanism 226 being placed in the stowed configuration, which can be by way of the arm 230 being biased toward engagement as described herein. The compartment 234 can include a surface 238 that can engage with the arm 230. Some examples of the surface 238 can include a groove, channel, tab, flange, etc. In some embodiments, the surface 238 is on the push-to-release mechanism 226. As described herein, the button 232 can be manipulated via pressing, pulling, sliding, and/or twisting such that the arm 230 disengages with the surface 238, compartment 234, and/or push-to-release mechanism 226 to allow deployment of the push-to-release mechanism 226 by way of one or more springs of the push-to-release mechanism 226. In some embodiments, a cover 212 can enclose the compartment 234 in the receptacle 202 as described herein.

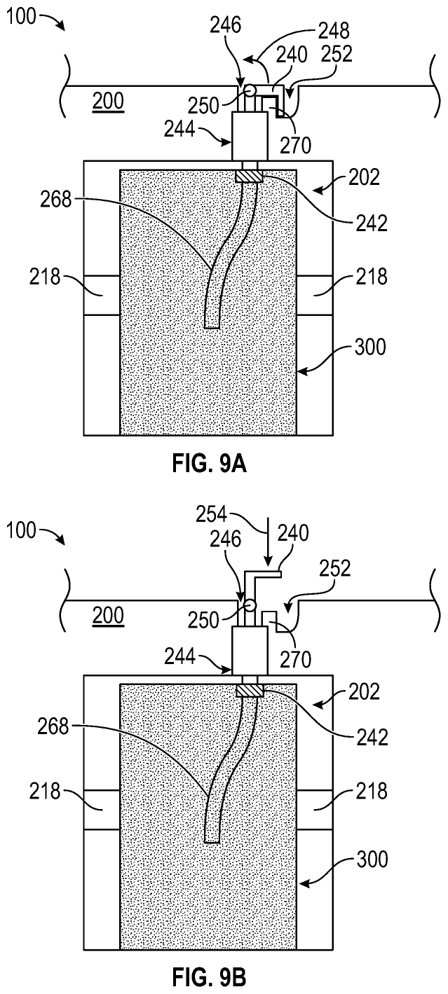

FIG. 7 shows an example of the sanitizing luggage 100 wherein a dispenser 240 facilitates dispensing a sanitizer from the sanitizer container 300 while positioned in the receptacle 202—e.g., in situ. The dispenser 240 can extend outside the receptacle 202 to be accessible to a user. The dispenser 240 can be fluidically coupled with the sanitizer container 300 such that the dispenser 240 can dispense sanitizer from within the sanitizer container 300 while the sanitizer container 300 is disposed in the receptacle 202. This can advantageously enable the user to dispense sanitizer without removing the sanitizer container 300 from the receptacle 202, which can enable the user to quickly cleanse the user's hands.

The dispenser 240 can include one or more spouts, pumps, tubes, pivots, springs, threads, etc. to facilitate dispensing. The sanitizing luggage 100 can incorporate a cover 212 that can impede and allow access into the receptacle 202, which can be beneficial for replacing the sanitizer container 300, replacing the sanitizer in the sanitizer container 300, and/or for air travel inspections. In some embodiments, the dispenser 240 can be moved between a stowed configuration and deployed configuration. In the stowed configuration, the dispenser 240 may not be configured to dispense sanitizer. In the deployed configuration, the dispenser 240 may be configured to dispense sanitizer. In some embodiments, the dispenser 240 can be manipulated, e.g., pushed, while in the deployed configuration to pump sanitizer from the sanitizer container 300 for dispensing.

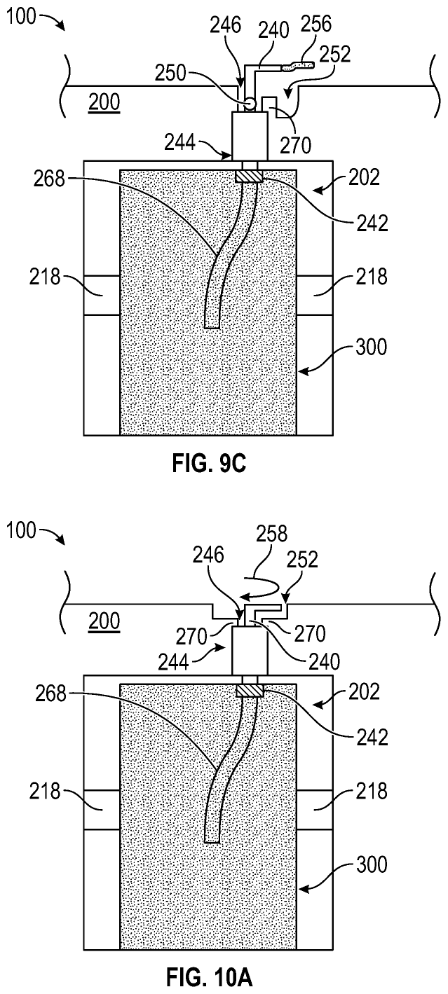

FIG. 8 shows an example of the sanitizing luggage 100 wherein the receptacle 202 is disposed between the arms 207 of a handle 206 of the luggage 200, as described in reference to FIG. 2. The sanitizing luggage 100 can include the dispenser 240. The dispenser 240 can extend from the sanitizer container 300 to outside the receptacle 202. The dispenser 240 can be fluidically coupled to the sanitizer container 300 while in the receptacle 202 such that sanitizer in the sanitizer container 300 can be dispensed to the user outside the receptacle 202. In some embodiments, the dispenser 240 is rigid. In some embodiments, the dispenser 240 can be rotated to different positions. In some variants, the dispenser 240 is flexible and/or includes one or more flexible features such that the user can manipulate the dispenser 240 to dispense in different directions.

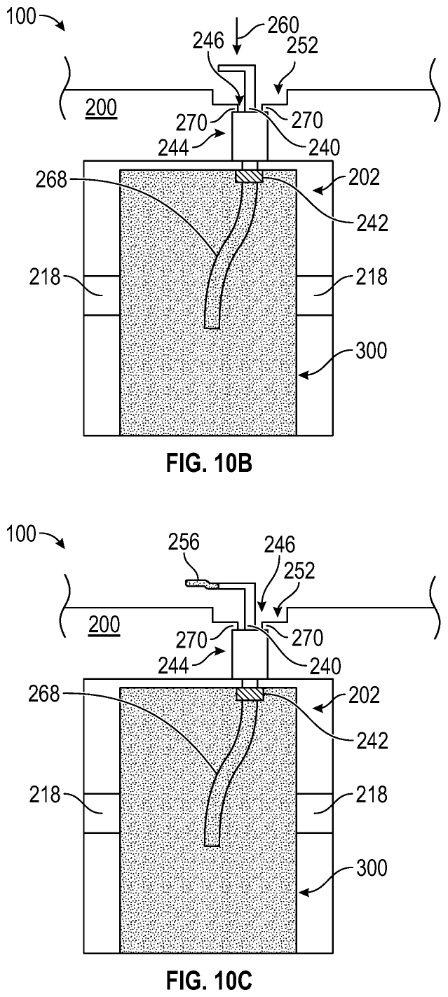

FIG. 9A-9C show an example of the sanitizing luggage 100 wherein the dispenser 240 can be rotated between stowed and deployed configurations. The dispenser 240 can extend through an opening 246 to outside of the receptacle 202. The dispenser 240 can be fluidically coupled to tubing 268, e.g., a tube, fluid line, etc., that extends into the sanitizer container 300 by way of a port 242, e.g., opening, conduit, etc., of the sanitizer container 300. The port 242 can include a seal, such as an o-ring or gasket, to prevent or reduce leakage of the sanitizer from within the sanitizer container 300. The dispenser 240 can be operatively coupled to a pump assembly 244, such that sanitizer can be pumped from within the sanitizer container 300 by way of the tubing 268 to outside the receptacle 202 through the dispenser 240. In some embodiments, the receptacle 202 can include one or more retainers 218, as described herein. In some embodiments, the receptacle 202 does not include one or more retainers 218.

FIG. 9A illustrates the dispenser 240 in the stowed configuration. In the stowed configuration, the dispenser 240 may be rotated into a recess 252, which may also be referred to as a gap, groove, void, etc., for stowage. The rotation of the dispenser 240 into the recess 252 can prevent inadvertent dispensing and/or catching of the dispenser 240 on objects. In the stowed configuration, the user may be prevented from dispensing sanitizer. For example, the rotation of the dispenser 240 to the stowed configuration can disrupt a fluid line between the dispenser 240 and tubing 268. In some embodiments, a stop 270 can prevent the dispenser 240 from being pushed inward to pump sanitizer from the sanitizer container 300. The stop 270 can be a protuberance, protrusion, or shelf that can impede motion of the dispenser 240 downward, such as rotation in a direction that is opposite of the arrow 248.

To dispense sanitizer, the dispenser 240 can be rotated by way of a pivot 250 in the direction of arrow 248 to a deployed configuration—an example of which is illustrated in FIG. 9B. In some embodiments, the pivot 250 can impede rotation of the dispenser 240 past the deployed configuration in the direction of arrow 248. In some embodiments, the dispenser 240 can be vertically oriented in the deployed configuration. In the deployed configuration, the fluid line between the dispenser 240 and tubing 268 can be connected for flow and/or the dispenser 240 can be rotated free of the impeding stop 270. A force can then be applied to the dispenser 240 in the direction of arrow 254 causing the dispenser 240 to translate or move in the direction of arrow 254 and the pump assembly 244 to move sanitizer from within the sanitizer container 300, through the tubing 268, and out the dispenser 240, as illustrated in FIG. 9C wherein sanitizer 256 is flowing out of the dispenser 240. Upon ceasing to apply a force in the direction of arrow 254, the dispenser 240 can return to the deployed configuration in FIG. 9B, which can be facilitated via the pump assembly 244. The user can then rotate the dispenser 240 back to the stowed configuration by way of rotating the dispenser 240 in the direction opposite of the arrow 248.

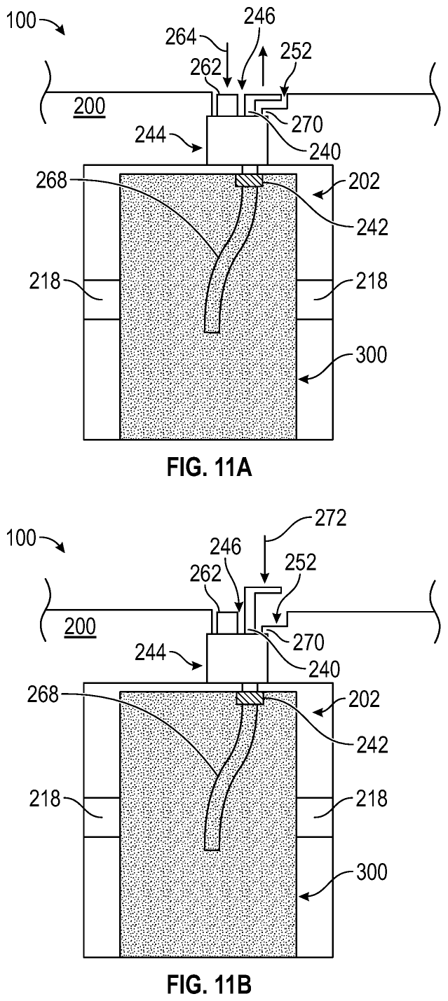

FIGS. 10A-10C show an example of the sanitizing luggage 100 wherein the dispenser 240 can be twisted between deployed and stowed configurations. FIG. 10A illustrates the dispenser 240 in the stowed configuration. In the stowed configuration, the dispenser 240 can be disposed in a recess 252, which can prevent inadvertent dispensing and/or catching of the dispenser 240 on objects. In some embodiments, the stop 270 can prevent the dispenser 240 from being pushed downward to pump sanitizer from the sanitizer container 300.

To dispense sanitizer, the dispenser 240 can be twisted in the direction of arrow 258 to a deployed configuration—an example of which is illustrated in FIG. 10B. In some embodiments, the recess 252 can permit twisting of the dispenser 240. In some embodiments, the dispenser 240 and pump assembly 244 can have a threaded connection that is disengaged upon the user twisting the dispenser 240 in the direction of arrow 258. Once disengaged, the pump assembly 244 can deploy the dispenser 240, translating the dispenser 240 away from the pump assembly 244. With the dispenser 240 deployed, a force can be applied to the dispenser 240 in the direction of arrow 260 causing the dispenser 240 to translate or move in the direction of arrow 260 and the pump assembly 244 to move sanitizer from within the sanitizer container 300, through the tubing 268, and out the dispenser 240, as illustrated in FIG. 10C wherein sanitizer 256 is flowing out of the dispenser 240. Upon ceasing to apply a force in the direction of arrow 260, the dispenser 240 can return to the deployed configuration in FIG. 10B, which can be facilitated via the pump assembly 244. Alternatively, the user can twist the dispenser 240 back to the stowed configuration by way of twisting the dispenser 240 in the direction opposite of arrow 258 while continuing to apply a force in the direction of arrow 260, which can engage a threaded connection between the dispenser 240 and pump assembly 244 to lock the dispenser 240 in the stowed—an example of which is illustrated in FIG. 10A.

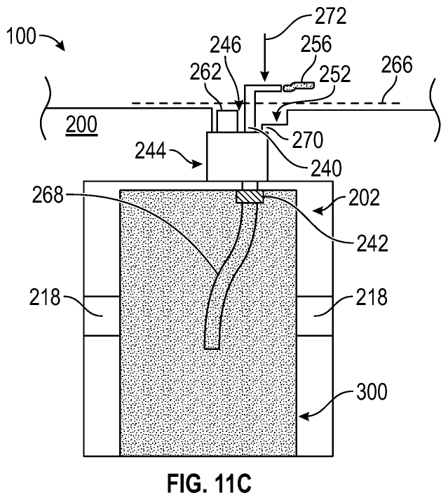

FIGS. 11A-11C show an example of the sanitizing luggage 100 wherein the dispenser 240 can be automatically moved from a stowed configuration to a deployed configuration by way of manipulating a button 262. FIG. 11A illustrates the dispenser 240 in the stowed configuration. In the stowed configuration, the dispenser 240 can be disposed in a recess 252, which can prevent inadvertent dispensing and/or catching of the dispenser 240 on objects. In some embodiments, the stop 270 can prevent or inhibit the dispenser 240 from being pushed downward to pump sanitizer from the sanitizer container 300.

To dispense sanitizer, a button 262 of the sanitizing luggage 100 can be manipulated to release the dispenser 240 for deployment. As described herein, the button 232 can be manipulated via pressing, pulling, sliding, and/or twisting. In some embodiments, the button 262 is part of the pump assembly 244. In some embodiments, the dispenser 240 is biased toward the deployed configuration, such as by way of the pump assembly 244, and a temporary fastener, such as a latch, clip, hook, clamp, clasp, catch, bracket, etc., of the pump assembly 244 that prevents or inhibits deployment when engaged. In some embodiments, manipulation of the button 262 disengages the latch, arm, clip, fastener, hook, clamp, clasp, catch, bracket, etc. of the pump assembly 244 allowing the dispenser 240 to automatically deploy to the deployed configuration—an example of which is illustrated in FIG. 11B. In the deployed configuration, a force can be applied to the dispenser 240 in the direction of arrow 272, causing the dispenser 240 to translate or move in the direction of arrow 254 and the pump assembly 244 to move sanitizer from within the sanitizer container 300, through the tubing 268, and out the dispenser 240, as illustrated in FIG. 11C wherein sanitizer 256 is flowing out of the dispenser 240. In some embodiments, the user can continue to apply a force to the dispenser 240 in the direction of arrow 272 until the dispenser 240 is once again in the stowed configuration—an example of which is illustrated in FIG. 11A. In some embodiments, the latch, clip, fastener, hook, clamp, clasp, catch, bracket, etc. of the pump assembly 244 automatically engages to retain the dispenser 240 in the stowed configuration. In some embodiments, the latch, clip, fastener, hook, clamp, clasp, catch, bracket, etc. of the pump assembly 244 automatically engages to retain the dispenser 240 in the stowed configuration once the dispenser 240 is positioned within the recess 252, which can be below the line 266. In some embodiments, the latch, clip, fastener, hook, clamp, clasp, catch, bracket, etc. of the pump assembly 244 engages to retain the dispenser 240 in the stowed configuration once the dispenser 240 is positioned within the recess 252 and the button 262 is manipulated.

In any of the embodiments described herein, the sanitizer of the sanitizing luggage 100 can be or can include a sanitization system for receiving and sanitizing a mobile electronic device, such as a smart phone. The sanitizing system can include a battery to power the operation of the sanitization system. The sanitization system can include a housing, a sanitizer contained within the housing configured to emit radiation with a sanitization effect, and/or an entry system with a default closed position, and/or any other structure, material, function, method or step that is described and/or illustrated in U.S. application Ser. No. 17/444,255, filed Aug. 2, 2021, which is hereby incorporated by reference in its entirety herein and made a part of this specification.

The following is claimed:

1. A luggage device comprising:
a receptacle comprising an opening, the receptacle configured to receive a sanitizer container through the opening;
a support configured to support the sanitizer container within the receptacle; and
a releasable holder that is configured to move the support between a deployed position in which the sanitizer container is at least partially exposed outside of the receptacle and a stowed position in which the sanitizer container is disposed inside the receptacle.

2. The luggage device of claim 1, further comprising a cover configured to cover the opening.

3. The luggage device of claim 2, further comprising a lock configured to secure the cover.

4. The luggage device of claim 2, further comprising a zipper configured to couple a periphery of the cover to a periphery of the opening.

5. The luggage device of claim 1, wherein the releasable holder is a push-to-release mechanism configured to automatically retain the support in the stowed position upon a pushing force translating the support to the stowed position from the deployed position.

6. The luggage device of claim 1, wherein the releasable holder is a push-to-release mechanism configured to automatically move the support to the deployed position from the stowed position upon a pushing force being applied to the support and then released.

7. The luggage device of claim 1, further comprising an arm configured to retain the support in the stowed position.

8. The luggage device of claim 7, further comprising a button that, upon manipulation, is configured to cause the arm to release the support such that the releasable holder moves the support to the deployed position.

9. The luggage device of claim 1, wherein the releasable holder comprises one or more springs configured to bias the support toward the deployed position and an arm configured to retain the support in the stowed configuration.

10. The luggage device of claim 1, further comprising a compartment configured to receive the sanitization container, the compartment being disposed on the support and configured to be placed inside the receptacle in the stowed position and at least partially outside the receptacle in the deployed position.

11. The luggage device of claim 1, further comprising one or more retainers disposed within the receptacle, the one or more retainers configured to secure the sanitizer container within the receptacle to prevent movement thereof.

12. The luggage device of claim 1, further comprising a handle with two arms, wherein the receptacle is disposed between the two arms of the handle.

13. A luggage device comprising:
a receptacle comprising an opening, the receptacle configured to receive a sanitizer container through the opening;
a dispenser configured to be fluidically coupled to the sanitizer container in the receptacle and to extend outside the receptacle; and
a pumping assembly, the pumping assembly operatively coupled to the dispenser and configured to pump a sanitizer in the sanitizer container out the dispenser upon an application of a pushing force on the dispenser;
wherein the dispenser is configured to be manipulated between a deployed configuration and a stowed configuration.

14. The luggage device of claim 13, wherein the dispenser comprises a pivot configured to enable the dispenser to be rotated between the stowed configuration and the deployed configuration.

15. The luggage device of claim 13, wherein the dispenser is configured to be retained in the stowed configuration via a threaded connection with the pump assembly, and wherein twisting of the dispenser is configured to disengage the threaded connection such that the pump assembly can automatically deploy the dispenser.

16. The luggage device of claim 13, further comprising a button, wherein manipulation of the button is configured to release the dispenser to automatically deploy by way of a biasing force from the pump assembly.

17. The luggage device of claim 14, further comprising an arm, wherein the manipulation of the button is configured to move the arm from retaining the dispenser in the stowed configuration.

18. The luggage device of claim 13, wherein the pumping assembly is manually actuated.

19. The luggage device of claim 13, wherein the pumping assembly is electronically actuated.

20. The luggage device of claim 13, further comprising a tube fluidically coupling the dispenser to the sanitizer container received in the receptacle.
